(19) United States
(12) Patent Application Publication
(10) Pub. No.: US 2006/0094700 A1
(43) Pub. Date: May 4, 2006

(54) Heat sterilization of a steroid in the presence of phosphate

(75) Inventor: Robert T. Lyons, Laguna Hills, CA (US)

Correspondence Address: ALLERGAN, INC., LEGAL DEPARTMENT, 2525 DUPONT DRIVE, T2-7H, IRVINE, CA, 92612-1599, US

(73) Assignee: ALLERGAN, INC., Irvine, CA (US)

(21) Appl. No.: 10/980,026

(22) Filed: Nov. 2, 2004

(51) Int. Cl. A61K 31/573

(52) U.S. Cl. 514/179

(57) ABSTRACT

A process for sterilizing a water-insoluble steroid composition comprising heat sterilizing the steroid in the presence of phosphate is disclosed herein. Compositions related thereto are also disclosed.

### DESCRIPTION OF RELATED ART

Among the therapies currently being practiced to treat ocular posterior segment disorders, such as uveitis, macular degeneration, macular edema and the like, is intravitreal injection of a corticosteroid, such as triamcinolone acetonide (TA). See, for example, Billson et al U.S. Pat. No. 5,770,589, the disclosure of which is incorporated in its entirety herein by reference. One medication commonly employed for this ophthalmic therapy is Kenalog® 40.

Steroid drug suspensions intended for parenteral administration are routinely heat sterilized since ultrafiltration is not an option. Change in particles size during the heat filtration process is often problematic. ZA 665331 discloses
Although drugs in the solid form and sparingly soluble in H₂O are not generally amenable to heat sterilization, heating suspensions of the drugs in H₂O saturated with NaCl is effective. The process is particularly suitable for steroids. To a partial suspension of 0.8 parts NaCl in 1.6 parts sterile H₂O heated to boiling and cooled was added 0.8 parts dexamethasone acetate (I) in jetomized form and 0.0075 parts poly(oxyethylene) (20) sorbitan monooleate (II). The mixture was sterilized by autoclaving at 121° 20-30 min. A solution of 0.9 parts PhCH₂OH, 0.0675 parts II, 0.5 parts Na CM-cellulose, and 70 parts H₂O was filtered, sterilized at 121° 15 min., cooled, combined aseptically with the foregoing suspension, and diluted to 100 parts with sterile H2O. The I crystals showed no change of particle size. The process was applied also to prednisolone tert-butylacetate, indomethacin, and tiabendazole.

EP1172098 discloses “an autoclavable ophthalmic composition comprising an ophthalmically effective drug.” The '098 patent also discloses the following related to the drug “Strongly preferred are ketotifen and dexamethasone.” In relation to the buffer, the following is disclosed “[e]xamples of buffer substances are acetate, ascorbate, borate, hydrogen carbonate/carbonate, citrate, gluconate, lactate, phosphate, propionate and TRIS (tromethamine) buffers.”

U.S. Pat. No. 6,468,548, also published as WO09936055, EP0938896, and CA2315767, “describes an autoclavable ophthalmic composition comprising an ophthalmically effective drug.” The '548 patent further discloses that the compositions “comprise an ophthalmically effective ingredient and in particular ketotifen or dexamethasone”, and that “[b]uffers, tonicity enhancing agents and preservatives different from quaternary ammonium salts may be used in an ophthalmic composition of the present invention too. Examples of buffer substances are acetate, ascorbate, borate, hydrogen carbonate/carbonate, citrate, gluconate, lactate, phosphate, propionate and TRIS (tromethamine) buffers.”

### BRIEF DESCRIPTION OF THE DRAWING FIGURES

FIG. 1 shows the effect of additives on particle size distribution of triamcinolone acetonide aqueous suspensions during autoclaving.

### DESCRIPTION OF THE INVENTION

A process for sterilizing a water-insoluble steroid composition comprising heat sterilizing the steroid in the presence of phosphate is disclosed herein. Compositions related thereto are also disclosed.

A “water-insoluble steroid” is a steroid which is not completely dissolved at the concentration at which it is administered in an aqueous composition. Thus, depending upon the use and concentration, a steroid may be considered water-insoluble in one situation but not water-insoluble in another situation. While not intending to limit the scope of the invention in any way, typical steroids include estrogens; glucocorticoids; progestins; mineralocorticoids; corticosteroids, such as cortisone, hydrocortisone, prednisone, prednisolone, methylprednisone, triamcinolone, fluoromethalone, dexamethasone, medrysone, betamethasone, loteprednol, fluocinolone, flumethasone, or mometasone; and androgens such as testosterone, methyltestosterone, or danazol. Often steroids are administered as ester, acetal, or ketal prodrugs, many of which are water-insoluble. These prodrugs are also considered to be steroids. While not intending to limit the scope of the invention in any way, triamcinolone actetonide is a steroid which is water-insoluble in many compositions in which it is used therapeutically.

A “prodrug”, as generally understood in the art, is a compound which is converted to a therapeutically active compound in vivo after administration, and the term should be interpreted as broadly herein as is generally understood in the art. While not intending to limit the scope of the invention, conversion may occur by hydrolysis of an ester group or some other biologically labile group.

In the process disclosed herein, heat sterilization may be accomplished at any temperature which will kill the pathogens of importance to the application in which the composition is used. Such a determination can be made without undue experimentation by a person of ordinary skill in the art. In some heat sterilization processes, the composition is heated to a temperature of 100° C. or greater. In other processes, the composition is heated to a temperature of 121° C. or greater.

Heating is carried out for the length of time required to kill the pathogens of importance to the application in which the composition is used. Such a determination is well within the skill of a person of ordinary skill in the art. In some processes, the composition is maintained at or near the peak temperature for at least 30 minutes. In other processes, the composition is maintained at or near the peak temperature for about 45 minutes. In other processes, the composition is maintained at or near the peak temperature for more than 45 minutes.

The term “phosphate” refers to phosphoric acid and its salts in any combination, including as individual species. In other words, phosphate is phosphoric acid, monovalent salts of phosphate, divalent salts of phosphate, and trivalent salts of phosphate, individually or in any combination.

The concentration of phosphate used is that required to improve control of any property related to particle size, including particle size distribution, average particle sized, maximum particle size, minimum particle sized, mean particle size, median particle size, particle number, and the like. Such a determination can be made by a person of ordinary skill in the art. In some processes, the concentration of the phosphate in a composition being sterilized is greater than 0.1%. Some compositions comprise 0.6% sodium phosphate dibasic and 0.08% sodium phosphate monobasic. In other processes, the weight ratio of phosphate to steroid is at least 0.01. In other words, there is at least 1 mg of phosphate for every 100 mg of steroid. In other processes, the weight ratio of phosphate to steroid is at least 0.03.

In some processes, the steroid is heat sterilized in a composition comprising both phosphate and sodium chloride. Some compositions comprise 20% triamcinoline actetonide, 1.58% sodium chloride, 0.75% sodium phosphate dibasic, and 0.10% sodium phosphate monobasic.

While not intending to limit the scope of the invention in any way, steroids are often used for the treatment or prevention of conditions affecting the eye. Some compositions prepared by the processes disclosed herein may be used for topical ophthalmic purposes. Some compositions prepared by the processes disclosed herein may be useful for injection into the eye.

Certain compositions comprise a water-soluble polymer. While not intending to limit the scope of the invention in any way, cellulose derivatives such as carboxymethylcellulose and hydroxypropylmethylcellulose are useful water-soluble polymers for certain of the compositions disclosed herein.

While not intending to limit the scope of the invention in any way, topical ophthalmic formulations often comprises an effective amount of buffer necessary to maintain the pH at the desired range, one or more tonicity agents, a preservative, and a chelating agent.

Buffers are well known by those skilled in the art and some examples of useful buffers are acetate, borate, carbonate, citrate, histidine, and phosphate buffers. While not intending to limit the scope of the invention in any way, certain compositions disclosed herein have a pH of from 4 to 8. Other compositions have a pH of from 4.5 to 5.5.

Tonicity agents are used to adjust the composition of the formulation to the desired isotonic range. Tonicity agents are well known in the art and some examples include glycerin, mannitol, sorbitol, sodium chloride, and other electrolytes.

Preservatives are used to prevent microbial contamination in multiple-use preparations. Preservatives are well known in the art, and, while not intending to be limiting, examples include polyhexamethylenebiguanidine (PHMB), benzalkonium chloride (BAK), stabilized oxychloro complexes (otherwise known as Purite®), phenylmercuric acetate, chlorobutanol, sorbic acid, chlorhexidine, benzyl alcohol, parabens, thimerosal, and mixtures thereof.

A chelating agent is often used in ophthalmic compositions to enhance preservative effectiveness. The term “chelating agent” has the meaning generally understood in the art, and while not intending to be limiting, suitable chelating agents include edetate salts like edetate disodium, edetate calcium disodium, edetate sodium, edetate trisodium, and edetate dipotassium.

The best mode of making and using the present invention are described in the following examples. These examples are given only to provide direction and guidance in how to make and use the invention, and are not intended to limit the scope of the invention in any way.

### EXAMPLE 1

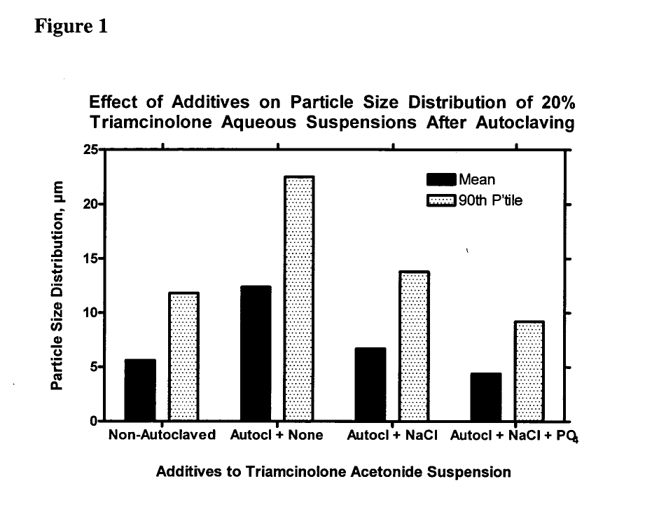

The compositions of FIG. 1 were prepared by the following procedure. Triamcinolone acetonide slurries were 20% w/w. These were prepared either in distilled water, water containing 1.58% sodium chloride, or water containing 1.58% sodium chloride plus 0.75% dibasic sodium phosphate (heptahydrate) and 0.10% monobasic sodium phosphate (monohydrate). All slurries were adjusted to pH 7.3 and then transferred to 10 mL pharmaceutical glass vials fitted with rubber stoppers and aluminum overseals. Autoclaving was accomplished in a bench top steam autoclave at 121° C. for 45 minutes (Brinkmann 2540E). Following autoclaving, all suspensions were stirred gently with a magnetic stir bar for about 16 hours at room temperature to disrupt any loose aggregates of drug crystals. Particle size distributions (mean and 90^{th} percentile) were then measured by laser light scattering (Beckman Coulter LS230).

While not intending to be bound by theory, or to limit the scope of the invention in any way, the results presented in FIG. 1 show that phosphate salts are effective in controlling particle size. Compared with control (“Non-Autoclaved”), the formulation autoclaved without sodium chloride or phosphate salts (“Autoc+None”) experienced a 91% increase in particle size (90^{th} percentile). The formulation that contained only sodium chloride experienced a significant, though smaller increase in particle size after autoclaving. However, the formulation containing both NaCl and phosphate salts experienced a significant decrease in particle size after autoclaving. While not intending to limit the scope of the invention or be bound in any way by theory, this result suggests the breakup of some crystal aggregates has occurred. While not intending to limit the scope of the invention in any way, this is particularly important for injectable suspensions, where larger particles sediment rapidly and can contribute to blockage of the fine gauge needle.

### EXAMPLE 2

As described in Example 1, 20% triamcinolone acetonide slurry is made by combining triamcinolone acetonide crystals with water, sodium chloride, and phosphate buffer salts, followed by bulk heat sterilization (autoclaving). This slurry is then blended under aseptic conditions with a sterile 5% hydrogel composed of high molecular weight (about 106 Daltons) sodium hyaluronate polymer. Water is added q.s. and the viscous mixture blended at controlled room temperature and under low shear conditions until homogenous. The resulting 8% triamcinolone acetonide gel suspension is suitable for intravitreal injection. Small particle size (about 5 μm mean diameter) for the steroid when autoclaved as described herein, combined with high viscosity of the hydrogel medium, enables the suspension to retain content uniformity with respect to triamcinolone acetonide distribution for up to two years.

### EXAMPLE 3

The product of Example 2 is injected into the vitreous humor of a person suffering from conditions requiring long-term ocular steroid administration such as uveitis, macular edema secondary to retinal vascular disease, or choroidal neovascularization associated with age-related macular degeneration. Deterioration of visual acuity is delayed, stopped, or acuity is improved over time.

What is claimed is:

1. A sterile composition comprising a water-insoluble steroid, said composition being prepared by a process comprising heat sterilizing the steroid in the presence of phosphate.

2. The composition of claim 1 wherein said composition is intended for treatment or prevention of a condition affecting an eye.

3. The composition of claim 2 wherein the steroid is triamcinolone or a prodrug thereof.

4. The composition of claim 3 wherein the steroid is triamcinolone acetonide.

5. The composition of claim 1 wherein the heating is to a temperature of 100° C. or greater.

6. The composition of claim 5 wherein the heating is to a temperature of about 121° C. or greater for at least 30 minutes.

7. The composition of claim 5 wherein the heating is to a temperature of about 121° C. for about 45 minutes, and wherein the steroid is triamcinolone acetonide.

8. The composition of claim 7 wherein the steroid is heated in the presence of sodium chloride and phosphate.

9. A process for sterilizing a water-insoluble steroid composition comprising heat sterilizing the steroid in the presence of phosphate.

10. The process of claim 9 wherein greater than 0.1% phosphate is present.

11. The process of claim 9 wherein the weight ratio of phosphate to steroid is at least 0.01.

12. The process of claim 11 wherein the weight ratio of phosphate to steroid is at least 0.03.

13. The process of claim 10 wherein about 0.75% sodium phosphate dibasic and about 0.10% sodium phosphate monobasic is present.

14. The composition of claim 7 comprising 20% triamcinolone acetonide, 1.58% sodium chloride, 0.75% sodium phosphate dibasic, and 0.10% sodium phosphate monobasic.

15. The composition of claim 7 wherein said process further comprises formulating the steroid for injection into an eye.

16. The composition of claim 14 wherein said process further comprises formulating the steroid for injection into an eye.
